(19) United States
(12) Patent Application Publication
(10) Pub. No.: US 2020/0350065 A1
(43) Pub. Date: Nov. 5, 2020

(54) ASSISTANCE SYSTEM, ASSISTANCE METHOD, ASSISTANCE PROGRAM, AND RECORDING MEDIUM ON WHICH ASSISTANCE PROGRAM IS RECORDED

(71) Applicant: TERUMO KABUSHIKI KAISHA, Tokyo (JP)

(72) Inventors: Takito INUKAI, Shizuoka (JP); Yasushi KINOSHITA, Shizuoka (JP); Yoshinobu ISAKA, Kanagawa (JP); Ryuta MIYASAKA, Shizuoka (JP)

(73) Assignee: TERUMO KABUSHIKI KAISHA, Tokyo (JP)

(21) Appl. No.: 16/935,304

(22) Filed: Jul. 22, 2020

Related U.S. Application Data
(63) Continuation of application No. PCT/JP2018/028721, filed on Jul. 31, 2018.

(30) Foreign Application Priority Data
Feb. 1, 2018 (JP) 2018-016294

(51) Int. Cl. G16H 40/20; G16H 10/60; G16H 20/40; G06N 5/04

(52) U.S. Cl. CPC G16H 40/20; G06N 5/04; G16H 20/40; G16H 10/60

(57) ABSTRACT

An assistance system capable of efficiently selecting a proper doctor from a plurality of doctors in accordance with physical conditions of the doctors and surgery difficulties on a scheduled surgery date. An assistance system includes an information acquisition unit that acquires disease information, lesion area information, and a scheduled surgery date of a scheduled surgery, a lesion area evaluation unit that uses surgery information on a past similar surgery having the lesion area information similar to that of the scheduled surgery, a doctor evaluation unit that uses surgery information on the same past disease surgery performed by each of the plurality of doctors and having at least disease information coinciding with that of the scheduled surgery, and a proposal unit that proposes the doctor whose doctor score falls within a range based on the lesion area score, as a surgeon-in-charge for the scheduled surgery.

### CROSS-REFERENCES TO RELATED APPLICATIONS

This application is a continuation of International Application No. PCT/JP2018/028721 filed on Jul. 31, 2018, which claims priority to Japanese Application No. 2018-016294 filed on Feb. 1, 2018, the entire content of both of which is incorporated herein by reference.

### FIELD OF THE DISCLOSURE

The present disclosure generally relates to an assistance system, an assistance method, an assistance program, and a recording medium on which an assistance program is recorded, which assist selection of a surgeon-in-charge for a scheduled surgery.

### BACKGROUND DISCUSSION

In recent years, a system for automatically carrying out work relating to medical business has been developed. For example, Japanese Patent Application Publication No. 2010-97572 discloses a system which determines each surgery time of a plurality of surgeries so that surgery times do not overlap each other when the plurality of surgeries are performed on the same day. According to this system, work relating to the surgeries can be efficiently carried out.

However, the system disclosed in Japanese Patent Application No. 2010-97572 assists work for determining a surgery time, but does not assist work for selecting, for example, a proper surgeon-in-charge from a plurality of doctors. For example, in a case where a relatively low-skilled doctor is in charge of a relatively difficult surgery, there is a possibility that the surgery time may be prolonged or a surgery success rate may decrease. In a case when a relatively high-skilled doctor is in charge of a relatively easy surgery, there is a possibility that the relatively high-skilled doctor may not be able to perform a relatively difficult surgery. In a case when the relatively high-skilled doctor's physical condition is relatively poor (i.e., not feeling well), there is a possibility that the doctor may be unable to bring out his or her own performance during the surgery. Therefore, it may be desirable to efficiently select a proper surgeon-in-charge for a scheduled surgery from a plurality of doctors in accordance with physical conditions of the doctors and surgery difficulties on a scheduled surgery date.

### SUMMARY

An assistance system, an assistance method, an assistance program, and a recording medium having an assistance program recorded on the recording medium, which are capable of efficiently selecting a proper surgeon-in-charge for a scheduled surgery from a plurality of doctors in accordance with physical conditions of the doctors and surgery difficulties on a scheduled surgery date.

In accordance with an aspect, an assistance system is disclosed for assisting selection of a surgeon-in-charge from a plurality of doctors. The assistance system includes an information acquisition unit that acquires disease information, lesion area information, and a scheduled surgery date of a scheduled surgery, a lesion area evaluation unit configured to acquire surgery information on a past similar surgery having the lesion area information similar to that of the scheduled surgery, configured to evaluate a difficulty in the similar surgery by using the surgery information on the past similar surgery, and configured to calculate a lesion area score which predicts a difficulty in the scheduled surgery, based on an evaluation result of the difficulty in the similar surgery, a doctor evaluation unit configured to acquire surgery information on the same past disease surgery previously performed by each of the plurality of doctors and having at least disease information coinciding with that of the scheduled surgery, and each work schedule of the plurality of doctors, and configured to calculate a doctor score which predicts capability of each of the plurality of doctors at the scheduled surgery date by using the surgery information on the same past disease surgery and the work schedule, and a proposal unit configured to propose the doctor whose doctor score falls within a range of an upper limit value and a lower limit value based on the lesion area score, as the surgeon-in-charge for the scheduled surgery.

In accordance with another aspect, an assistance method is disclosed for assisting selection of a surgeon-in-charge from a plurality of doctors. The assistance method includes acquiring disease information on a scheduled surgery, lesion area information, and a scheduled surgery date, acquiring surgery information on a past similar surgery having the lesion area information similar to that of the scheduled surgery, evaluating a difficulty in the similar surgery by using the surgery information on the past similar surgery, and calculating a lesion area score which predicts a difficulty in the scheduled surgery, based on an evaluation result of the difficulty in the similar surgery, acquiring surgery information on the same past disease surgery previously performed by each of the plurality of doctors and having at least disease information coinciding with that of the scheduled surgery, and each work schedule of the plurality of doctors, and calculating a doctor score which predicts capability of each of the plurality of doctors at the scheduled surgery date by using the surgery information on the same past disease surgery and the work schedule, and proposing the doctor whose doctor score falls within a range of an upper limit value and a lower limit value based on the lesion area score, as the surgeon-in-charge for the scheduled surgery.

In accordance with a further aspect, an assistance program is disclosed for assisting selection of a surgeon-in-charge from a plurality of doctors. The assistance program includes a procedure of acquiring disease information on a scheduled surgery, lesion area information, and a scheduled surgery date, a lesion area evaluation procedure of acquiring surgery information on a past similar surgery having the lesion area information similar to that of the scheduled surgery, evaluating a difficulty in the similar surgery by using the surgery information on the past similar surgery, and calculating a lesion area score which predicts a difficulty in the scheduled surgery, based on an evaluation result of the difficulty in the similar surgery, a procedure of acquiring surgery information on the same past disease surgery previously performed by each of the plurality of doctors and having at least disease information coinciding with that of the scheduled surgery, and each work schedule of the plurality of doctors, and calculating a doctor score which predicts capability of each of the plurality of doctors at the scheduled surgery date by using the surgery information on the same past disease surgery and the work schedule, and a procedure of proposing the doctor whose doctor score falls within a range of an upper limit value and a lower limit value based on the lesion area score, as the surgeon-in-charge for the scheduled surgery.

In accordance with another aspect, a non-transitory computer readable medium (CRM) storing computer program code executed by a computer processor that executes a process for assisting selection of a surgeon-in-charge from a plurality of doctors, the process comprising: acquiring disease information on a scheduled surgery, lesion area information, and a scheduled surgery date; acquiring surgery information on a past similar surgery having the lesion area information similar to that of the scheduled surgery, evaluating a difficulty in the similar surgery by using the surgery information on the past similar surgery, and calculating a lesion area score which predicts a difficulty in the scheduled surgery, based on an evaluation result of the difficulty in the similar surgery; acquiring surgery information on the same past disease surgery previously performed by each of the plurality of doctors and having at least disease information coinciding with that of the scheduled surgery, and each work schedule of the plurality of doctors, and calculating a doctor score which predicts capability of each of the plurality of doctors at the scheduled surgery date by using the surgery information on the same past disease surgery and the work schedule; and proposing the doctor whose doctor score falls within a range of an upper limit value and a lower limit value based on the lesion area score, as the surgeon-in-charge for the scheduled surgery.

In accordance with an aspect, the lesion area score which predicts the difficulty in the scheduled surgery is calculated using the surgery information on the past similar surgery. According to the present disclosure, the doctor score which predicts the capability of the doctor on the scheduled surgery date is calculated using the surgery information on the same past disease surgery and the work schedule. The doctor whose doctor score falls within the range of the upper limit value and the lower limit value based on the lesion area score is proposed as the surgeon-in-charge. That is, the doctor whose doctor score (capability on the scheduled surgery date) falls within a prescribed range with respect to the lesion area score (difficulty in the surgery) is proposed as the surgeon-in-charge. Therefore, according to the present disclosure, a proper doctor can be efficiently selected from the plurality of doctors in accordance with the physical conditions of the doctors and the surgery difficulties on the scheduled surgery date.

### BRIEF DESCRIPTION OF THE DRAWINGS

FIG. 1 is a view illustrating a schematic configuration of an assistance system according to an embodiment disclosed here.

FIG. 2A is a block diagram illustrating a hardware configuration of the assistance system according to an embodiment disclosed here.

FIG. 2B is a block diagram illustrating a functional configuration of the assistance system according to an embodiment disclosed here.

FIG. 3A is a view illustrating past surgery information of the assistance system according to an embodiment disclosed here.

FIG. 3B is a view illustrating a work schedule of the assistance system according to an embodiment disclosed here.

FIG. 4A is a view for describing classification of the past surgery information of the assistance system according to an embodiment disclosed here.

FIG. 4B is a view for describing classification of the past surgery information of the assistance system according to an embodiment disclosed here.

FIG. 5 is a flowchart illustrating an assistance method according to an embodiment disclosed here.

FIG. 6A is a subroutine indicating a lesion area evaluation step in FIG. 5.

FIG. 6B is a subroutine indicating a doctor evaluation step in FIG. 5.

FIG. 6C is a subroutine indicating a proposal step in FIG. 5.

FIG. 7A is a view illustrating contents displayed on a display of an intra-hospital terminal in an information acquisition step in FIG. 5.

FIG. 7B is a view illustrating contents displayed on the display of the intra-hospital terminal in the proposal step in FIG. 5.

### DETAILED DESCRIPTION

Set forth below with reference to the accompanying drawings is a detailed description of embodiments of an assistance system, an assistance method, an assistance program, and a recording medium having an assistance program recorded on the recording medium, which assist selection of a surgeon-in-charge representing examples of the inventive assistance system, the assistance method, the assistance program, and the recording medium having the assistance program recorded on the recording medium, which assist selection of a surgeon-in-charge disclosed here. In the description of the drawings, the same reference numerals will be assigned to the same elements, and repeated description will be omitted. Dimensional ratios in the drawings are exaggerated for convenience of the description, and may be different from actual ratios in some cases.

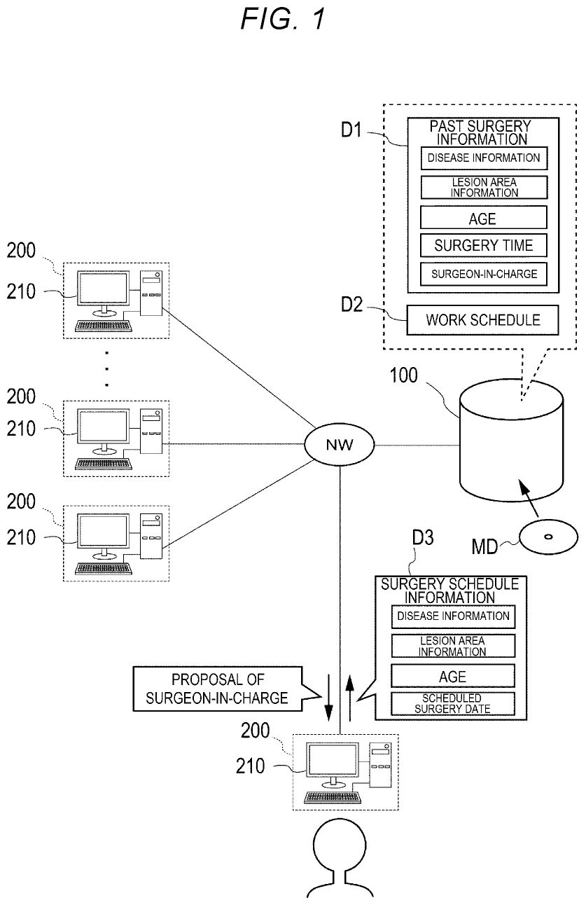

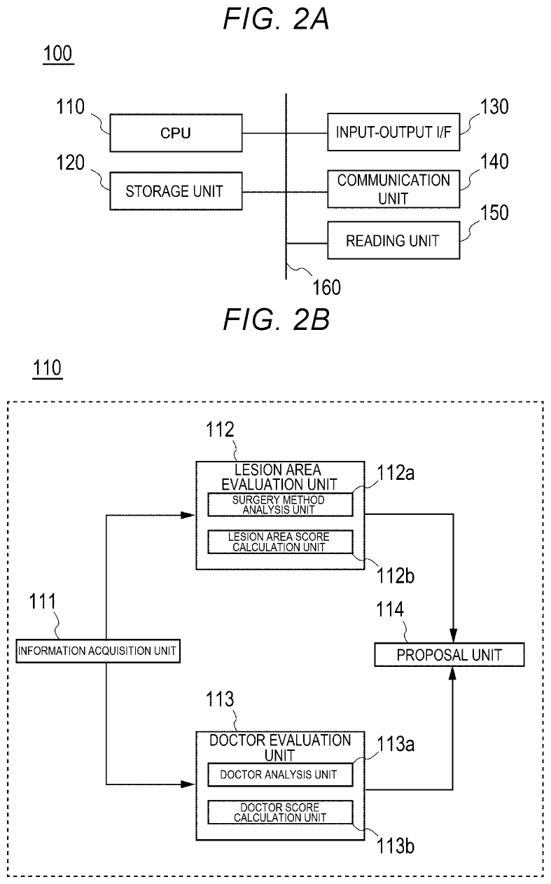

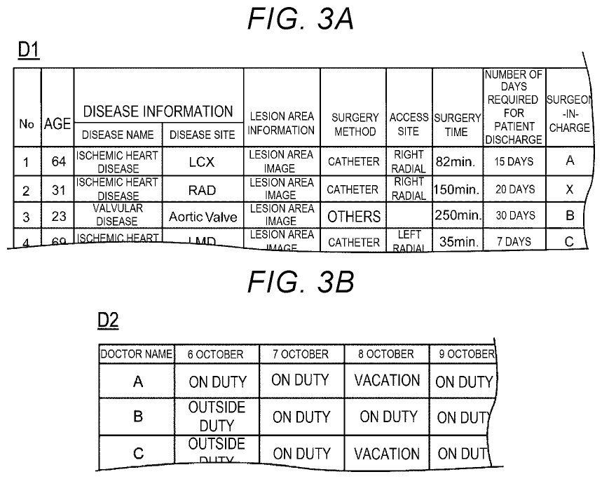

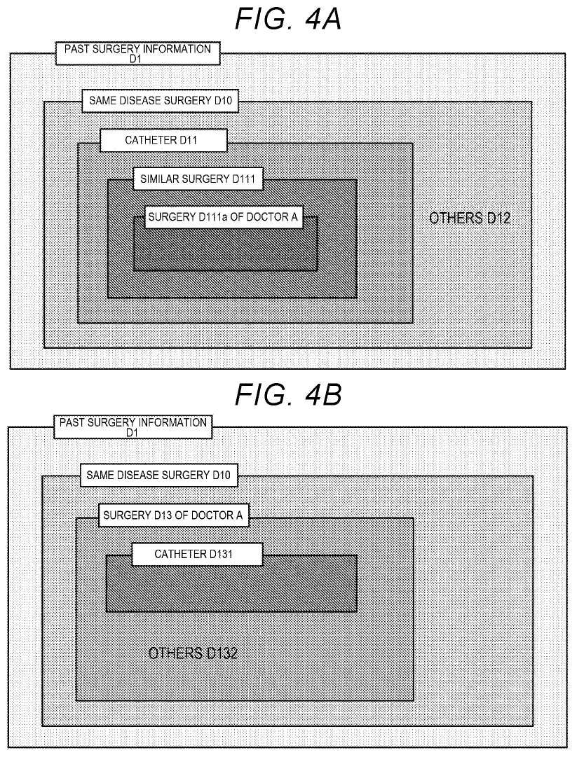

FIG. 1 is a view for describing an overall configuration of an assistance system 100 according to an embodiment disclosed. FIGS. 2A and 2B are views for describing each unit of the assistance system 100. FIGS. 3A to 3C, 4A, and 4B are views for describing information handled by the assistance system 100.

As illustrated in FIG. 1, the assistance system 100 proposes a proper doctor from a plurality of doctors inside a hospital (intra-hospital doctors), as a surgeon-in-charge, in accordance with physical conditions of the doctors and surgery difficulties on a scheduled surgery date.

The assistance system 100 can be connected to a plurality of intra-hospital terminals 200 via an intra-hospital network NW, and is configured to serve as a server that transmits and receives data between the intra-hospital terminals 200. A user of the assistance system 100 can operate one of the intra-hospital terminals 200 (operation terminal) to request the assistance system 100 to propose the surgeon-in-charge. Hereinafter, the assistance system 100 will be described in detail.

First, a hardware configuration of the assistance system 100 will be described.

As illustrated in FIG. 2A, the assistance system 100 can include a central processing unit (CPU) 110, a storage unit 120, an input-output I/F 130, a communication unit 140, and a reading unit 150. The CPU 110, the storage unit 120, the input-output I/F 130, the communication unit 140, and the reading unit 150 are connected to a bus 160, and exchange data with each other via the bus 160. Hereinafter, each unit will be described.

The CPU 110 is configured to control each unit and performs various arithmetic processes in accordance with various programs stored in the storage unit 120.

The storage unit 120 is configured to include a read only memory (ROM) for storing various programs or various data items, a random access memory (RAM) for temporarily storing programs or data as a work region, and a hard disk for storing various programs including an operating system or various data items.

Although not limited, the input-output I/F 130 is an interface for connecting an input device such as a keyboard and a mouse and an output device such as a display and a printer to each other, for example.

The communication unit 140 is an interface for communicating with the plurality of intra-hospital terminals 200.

The reading unit 150 is configured to read data recorded on a computer-readable recording medium MD (refer to FIG. 1). Although not limited, the computer-readable recording medium MD can be configured to include an optical disk such as a CD-ROM and a DVD-ROM, a USB memory, or an SD memory card, for example. Although not limited, the reading unit 150 can be configured to include a CD-ROM drive or a DVD-ROM drive, for example.

Next, a main function of the assistance system 100 will be described.

The storage unit 120 is configured to store an assistance program that proposes the proper doctor as the surgeon-in-charge from the plurality of doctors inside the hospital, in accordance with the physical conditions of the doctors and the difficulties in the scheduled surgery on the scheduled surgery date. According to the present embodiment, the assistance program is provided by the computer-readable recording medium MD.

The storage unit 120 is configured to store past surgery information D1 relating to surgeries previously performed by the intra-hospital doctor, and a work schedule D2 of the intra-hospital doctor.

As illustrated in FIG. 3A, for example, the past surgery information D1 can include information on a surgery number (described as “No” in the drawing), a patient age, disease information, lesion area information, a surgery method, an access site, a surgery time, and the number of days required for patient discharge, and a surgeon-in-charge (i.e., doctor-in-charge). The information is stored in the storage unit 120 in a state of being associated with each surgery. For example, the disease information can include a disease name and a disease site. The lesion area information is not limited as long as it is possible to recognize a dimension and a state of a lesion area (for example, in a case where a blood vessel is stenosed, a length of a stenosed site corresponds to the dimension, and a stenosed degree of the stenosed site corresponds to the state). For example, the lesion area information can be configured to include a lesion area image captured using X-ray, CT, and MRI methods. In FIG. 3A, in order to simplify the description, the surgery method can be, for example, classified into two types such as a surgery performed using a catheter (illustrated as “catheter” in the drawing) and a surgery performed using a method other than the catheter (illustrated as “others” in the drawing). However, the classification methods of the surgery method are not limited. In a case where the surgery method is a catheter, for example, an access site (puncture site) such as a radial artery of a right arm (illustrated as “right radial” in the drawing) and a radial artery of a left arm (illustrated as “left radial” in the drawing) can be included in the surgery information. The number of days required for patient discharge, for example, can be the number of days from a surgery date to a patient discharge date.

For example, as illustrated in FIG. 3B, the work schedule D2 is configured to include a schedule table indicating a work status (for example, outside duty, vacation, and on-duty) of the intra-hospital doctors for each day.

As illustrated in FIG. 2B, the CPU 110 is configured to function as an information acquisition unit 111, a lesion area evaluation unit 112, a doctor evaluation unit 113, and a proposal unit 114 by executing an assistance program stored in the storage unit 120. Hereinafter, each unit will be described.

First, the information acquisition unit 111 will be described.

As illustrated in FIG. 1, the information acquisition unit 111 is configured to acquire surgery schedule information D3 relating to a scheduled surgery from the intra-hospital terminal 200 operated by a user. For example, the surgery schedule information D3 can include disease information, lesion area information, an age of a patient having a scheduled surgery, and a scheduled surgery date. The disease information can include, for example, a disease name and a disease site, as in the past surgery information. The lesion area information is not limited, for example, as long as a dimension and a state of a lesion area can be recognized, as in the past surgery information. For example, the lesion area information can include a lesion area image captured using X-ray, CT, and MRI methods. The age of the patient having the scheduled surgery is the age of the patient on the scheduled surgery date. The scheduled surgery date is not limited to one day, and may be a plurality of days in a case where a plurality of candidate days are required.

As illustrated in FIG. 4A, the information acquisition unit 111 is configured to acquire surgery information D10 on the same past disease surgery having the disease information coinciding with that of the scheduled surgery (hereinafter, simply referred to as the “same disease surgery D10”), from past surgery information D1 stored in the storage unit 120. The acquired same disease surgery D10 can be used for processes performed by a lesion area evaluation unit 112 and a doctor evaluation unit 113 (to be described later).

Next, the lesion area evaluation unit 112 will be described.

As illustrated in FIG. 2B, the lesion area evaluation unit 112 functions as a surgery method analysis unit 112a configured to analyze a surgery method, and a lesion area score calculation unit 112b configured to calculate a lesion area score which predicts a difficulty in the scheduled surgery.

The surgery method analysis unit 112a classifies the same disease surgery D10 acquired by the information acquisition unit 111 for each surgery method, and calculates a ratio of the number of the same disease surgeries using each surgery method to the number of the same disease surgeries D10. Specifically, for example, as illustrated in FIG. 4A, the surgery method analysis unit 112a is configured to classify, for example, the same disease surgery D10 into the same disease surgery D11 using a catheter surgery method (hereinafter, simply referred to as the “same disease surgery D11 using a catheter”) and the same disease surgery D12 using other surgery methods (hereinafter, simply referred to as the “same disease surgery D12 using others”). The surgery method analysis unit 112a is configured to calculate a ratio of the number of the same disease surgeries D11 using the catheter to the number of the same disease surgeries D10, and a ratio of the number of the same disease surgeries D12 using others to the number of the same disease surgeries D10. Therefore, a proposal unit 114 (to be described later) can present a ratio of each surgery method in the same disease surgeries D10 to a user, and the user can use the presented information as a reference for selecting the surgery method.

The surgery method analysis unit 112a may classify the same disease surgery D11 using the catheter for each access site, and may calculate an average number of days required for patient discharge for each access site. As the average number of days required for patient discharge is less, the access site is a reduced burden on the patient. Therefore, the proposal unit 114 (to be described later) presents the access site to the user, sequentially from the shortest average number of days required for patient discharge, and the user uses the presented information as a reference for selecting the access site in a case where the surgery is performed using the catheter.

The lesion area score calculation unit 112b is configured to extract surgery information on a similar surgery having lesion area information similar to that of the scheduled surgery, from the surgery information of the same disease surgeries for each surgery method. The lesion area score calculation unit 112b is configured to compare an average surgery time of the same disease surgery with an average surgery time of the similar surgery for each surgery method. For example, when the surgery time is longer, it is considered that the difficulty in the surgery is relatively higher. Therefore, the average times can be compared with each other. In this manner, the lesion area score calculation unit 112b can evaluate the difficulty in the similar surgery for each surgery method. That is, the lesion area score calculation unit 112b can evaluate how difficult it is to perform the similar surgery in the same disease surgery, for each surgery method.

A case of using the catheter surgery method will be described as an example. The lesion area score calculation unit 112b is configured to acquire surgery information D111 of the similar surgery having the similar lesion area information (hereinafter, simply referred to as a “similar surgery D111 using the catheter”), from the same disease surgeries D11 using the catheter. The lesion area score calculation unit 112b compares an average surgery time of the same disease surgery D11 using the catheter with an average surgery time of the similar surgery D111 using the catheter. In this manner, the lesion area score calculation unit 112b can evaluate how difficult it is to perform the similar surgery D111 in the same disease surgery D11 using the catheter. For example, the difficulty in the similar surgery using the catheter can be defined as a ratio of the average surgery time of the similar surgery D111 using the catheter to the average surgery time of the same disease surgery D11 using the catheter, as expressed in Equation 1 below.

Difficulty in similar surgery using catheter=Average surgery time of similar surgery using catheter/Average surgery time of same disease surgery using catheter (Equation 1)

In an embodiment, similarity of the lesion area information means that a coincidence rate of the similarity of the lesion area information falls within a predetermined range in a case where the dimensions and the states of the lesion areas are compared with each other. For example, in a case where a blood vessel is stenosed, the similarity of the lesion area information means that the coincidence rate of the length of a stenosed site falls within a predetermined range, and the coincidence rate of a stenosed degree of the stenosed site falls within a predetermined range. The dimension and the state of the lesion area can be acquired by performing an image analysis on a lesion area image.

The lesion area score calculation unit 112b is configured to calculate a lesion area score which predicts a difficulty in the scheduled surgery, based on the difficulty in the similar surgeries using each surgery method. For example, in a case where the patient having the scheduled surgery is, for example, an infant or an elderly person, the difficulty of the scheduled surgery is predicted to increase. Therefore, in the present embodiment, the lesion area score calculation unit 112b calculates the lesion area score which predicts the difficulty in the scheduled surgery using each surgery method, based on the difficulty in the similar surgery using each surgery method and the age of the patient having the scheduled surgery. Specifically, for example, as the lesion area score, the lesion area score calculation unit 112b calculates a value obtained by multiplying the difficulty in the similar surgery using each surgery method by a weighting coefficient based on the age of the patient having the scheduled surgery. For example, the lesion area score in a case of using the catheter surgery method may be a value obtained by multiplying the difficulty in the similar surgery using the catheter by the weighting coefficient based on the age, as expressed in Equation 2 below.

Lesion area score=Weighting Coefficient based on age×Difficulty in similar surgery using catheter (Equation 2)

The weighting coefficient based on the age is not limited as long as influence of the difficulty in the surgery which is caused by the age can be reflected on the lesion area score. For example, the weighting coefficient based on the age can be set to a value greater than 1 in a case where the age of the patient having the scheduled surgery falls outside a range of an upper limit value and a lower limit value based on an average age of patients subjected to the same disease surgery using each surgery method. In Equation 2 above, in a case where the lesion area score is greater than 1, the difficulty in the scheduled surgery can be relatively high. In a case where the lesion area score is less than 1, the difficulty in the scheduled surgery can be relatively low. In a case where the lesion area score is approximately 1, the difficulty in the scheduled surgery can be, for example, standard (i.e., normal).

Next, the doctor evaluation unit 113 will be described.

As illustrated in FIG. 2B, the doctor evaluation unit 113 functions as a doctor analysis unit 113a configured to extract every doctor having surgery experience in the same disease surgery and having a vacant schedule on the scheduled surgery date, and configure to analyze the surgery method of the same disease surgery for each extracted doctor, and a doctor score calculation unit 113b configured to calculate a doctor score which predicts capability of each doctor on the scheduled surgery date.

The doctor analysis unit 113a acquires a work schedule D2, and extracts the doctor having the surgery experience in the same disease surgery D10 and having the vacant schedule on the scheduled surgery date.

The doctor analysis unit 113a is configured to extract the same disease surgery of the doctor having availability (i.e., not scheduled for another surgery) on the scheduled surgery date from the same disease surgeries D10, and classifies the same disease surgery for each surgery method. The doctor analysis unit 113a calculates a ratio of the number of the same disease surgeries using each surgery method to the number of the same disease surgeries for each available doctor on the scheduled surgery date. Specifically, for example, as illustrated in FIG. 4B, the doctor analysis unit 113a acquires the same disease surgery D13 of available doctor A on the scheduled surgery date, from the same disease surgeries D10. The doctor analysis unit 113a classifies the same disease surgery D13 of the doctor A into the same disease surgery using the catheter surgery method (hereinafter, referred to as the “same disease surgery D131 of the doctor A using the catheter”), and the same disease surgery using other surgery methods (hereinafter, referred to as the “same disease surgery D132 of the doctor A using others”). The doctor analysis unit 113a calculates a ratio of the number of the same disease surgeries D131 of the doctor A using the catheter to the number of the same disease surgeries D13 of the doctor A, and a ratio of the number of the same disease surgeries D132 of the doctor A using others to the number of the same disease surgeries D132 of the doctor A.

The doctor score calculation unit 113b is configured to acquire the similar surgery having the lesion area information similar to that of the scheduled surgery, from the same disease surgeries D10 acquired by the information acquisition unit 111. The doctor score calculation unit 113b classifies the acquired similar surgeries for each surgery method. The doctor score calculation unit 113b compares an average surgery time of the similar surgeries with an average time of the similar surgeries of each doctor for each surgery method. In accordance with an embodiment, a doctor is considered more skilled in the surgery when the surgery time of the doctor is shorter. Therefore, the average times can be compared with each other. In this manner, the doctor score calculation unit 113b can evaluate a skill level of each doctor in the similar surgery for each surgery method. Specifically, for example, as illustrated in FIG. 4A, the doctor score calculation unit 113b compares the average surgery time (of all doctors) in the similar surgeries D111 using the catheter with the average surgery time of the doctor A in the similar surgeries D111a using the catheter. In this manner, the doctor score calculation unit 113b can evaluate the skill level of the doctor A in the similar surgeries D111a using the catheter. For example, in the doctor score calculation unit 113b, the skill level of the doctor A in the similar surgeries using the catheter can be defined as a ratio of the average surgery time of the doctor A in the similar surgeries D111a using the catheter to the average surgery time in the similar surgeries D111 using the catheter, as expressed in Equation 3 below.

Skilled level of doctor A in similar surgery using catheter=Average surgery time of doctor A in similar surgeries using catheter/Average surgery time in similar surgeries using catheter (Equation 3)

Even if the doctor has a higher skill level in the similar surgeries, in a case where the doctor is not feeling well on the surgery date, there is a possibility that the doctor may be unable to exert his or her normal energy. Therefore, the doctor score calculation unit 113b calculates a doctor score which predicts capability of the doctor on the scheduled surgery date in view of the physical condition of the doctor on the scheduled surgery date in addition to the skill level of each doctor in the similar surgeries. In the present embodiment, the doctor score calculation unit 113b calculates a value obtained by multiplying the skill level of each doctor in the similar surgeries for each surgery method by the weighting coefficient based on the work schedule, as the doctor score which predicts the capability of each doctor on the scheduled surgery date. Specifically, the doctor score of the doctor A in a case of using the catheter surgery method can be defined as a value obtained by multiplying the weighting coefficient based on the work schedule by the skill level of the doctor A in the similar surgeries using the catheter, as expressed in Equation 4 below.

Doctor score of doctor A=Weighting coefficient based on work schedule×Skilled level of doctor A in similar surgeries using catheter (Equation 4)

The weighting coefficient based on the work schedule is not limited as long as influence on the capability of the doctor which is caused by the physical condition can be reflected on the doctor score. For example, the weighting coefficient based on the work schedule can be set to a value greater than 1. For example, in a case where the doctor continuously carries out work for a predetermined number of days or longer immediately before the scheduled surgery date, the physical condition of the doctor may become relatively poor due to fatigue on the scheduled surgery date, and it can be predicted that the capability of the doctor is lowered. In Equation 4 above, in a case where the doctor score is greater than 1, the skill level of the doctor is relatively low. In a case where the doctor score is less than 1, the skill level of the doctor is relatively high. In a case where the doctor score is approximately 1, the skill level of the doctor is standard.

In the present embodiment, the doctor score calculation unit 113b evaluates the skilled level of the doctor A by using the past similar surgeries D111a performed by the doctor A and having the lesion area information similar to that of the scheduled surgery. However, the past surgery information used by the doctor score calculation unit 113b to evaluate the skilled level of the doctor A is not limited as long as at least the disease information coincides with that of the scheduled surgery. For example, the doctor score calculation unit 113b may evaluate the skill level of the doctor A in the same disease surgeries, based on a comparison result between the average surgery time (of all doctors) in the same diseases surgeries using the catheter and the average surgery time of the doctor A in the same diseases surgeries using the catheter. The doctor score of the doctor A may be calculated, based on the skilled level in the same disease surgeries and the work schedule of the doctor A. For example, the doctor score calculation unit 113b may evaluate the skilled level of the doctor A in the similar surgeries, based on a comparison result between the average surgery time (of all doctors) in the same disease surgeries using the catheter and the average surgery time of the doctor A in the similar surgeries using the catheter. The doctor score of the doctor A may be calculated, based on the skilled level in the similar surgeries and the work schedule of the doctor A. However, in a case where many surgeries are performed for the same disease surgery, the doctor score calculation unit 113b can accurately evaluate the skilled level of the doctor for each lesion area of the same disease surgery. Therefore, as expressed in Equation 3 above, it is preferable to evaluate the skill level of the doctor A by using a ratio between the average surgery time of the doctor A in the similar surgeries using the catheter and the average surgery time in the similar surgeries using the catheter.

For example, the past surgery used in calculating the doctor score can be limited to surgery performed within a predetermined period. For example, when the past surgery used in calculating the doctor score is limited to the latest surgery, the recent skilled level of the doctor can be evaluated.

Next, the proposal unit 114 will be described.

For each surgery method, the proposal unit 114 is configured to extract the doctor whose doctor score falls within a range of the upper limit value and the lower limit value based on the lesion area score, as the surgeon-in-charge. For example, in a case where Equations 2 and 4 above are used, the upper limit value and the lower limit value can be set to a value to extract the doctor as follows. When the lesion area score is relatively high (when the difficulty is relatively high), the doctor having the relatively low doctor score (having relatively high capability) is the surgeon-in-charge. When the lesion area score is approximately medium (when the difficulty is approximately standard), the doctor having the approximately medium doctor score (having the approximately standard capability) is the surgeon-in-charge. When the lesion area score is relatively low (when the difficulty is relatively low), the doctor having the relatively high doctor score (having the relatively low capability) is the surgeon-in-charge. Specifically, for example, the upper limit value and the lower limit value can be set to a value to extract the doctor as follows. In a case where the lesion area score is 1.5 (when the difficulty is relatively high), the doctor having the doctor score of 0.2 to 0.5 (having the relatively high capability) is extracted. In a case where the lesion area score is 1.0 (when the difficulty is approximately standard), the doctor having the doctor score of 0.8 to 1.0 (having the approximately standard capability) is extracted. In a case where the lesion area score is 0.5 (when the difficulty is relatively low), the doctor having the doctor score of 1.2 to 1.5 (having the relatively low capability) is extracted.

The proposal unit 114 presents a ratio of each surgery method in the same disease surgeries D10 and a list of the doctors extracted as the surgeon-in-charge for each surgery method, to a user. In a case of using the catheter surgery method, the proposal unit 114 presents the access sites to the user, sequentially from the smallest number of days required for patient discharge. The user of the assistance system 100 can select the surgeon-in-charge from the presented list of the doctors. The proposal unit 114 notifies the selected doctor, for example, by email that he or she is the surgeon-in-charge, and registers a scheduled surgery in the work schedule of the selected doctor.

In this way, the assistance system 100 can automatically predict the physical condition of the doctor and the difficulty in the surgery on the scheduled surgery date, and can propose a proper doctor from the plurality of doctors for each surgery method. Therefore, the user can efficiently select the proper doctor. In this manner, for example, a work load on health care workers can be reduced. The doctor having the high capability is in charge of the surgery having the high difficulty. Therefore, a success rate of the surgery can be improved. The doctor having the low skill level can experience the surgery having the low difficulty. Accordingly, it is possible to improve the skill level by accumulating the experiences.

For example, in a case where the ratio of surgery using the catheter is high in the same disease surgeries D10, the proposal unit 114 may present the list of doctors extracted as the surgeon-in-charge in a case of using the catheter surgery method. In a case of using other surgery methods, the proposal unit 114 may simply present a predetermined number of the doctors having a high ratio of other surgery methods.

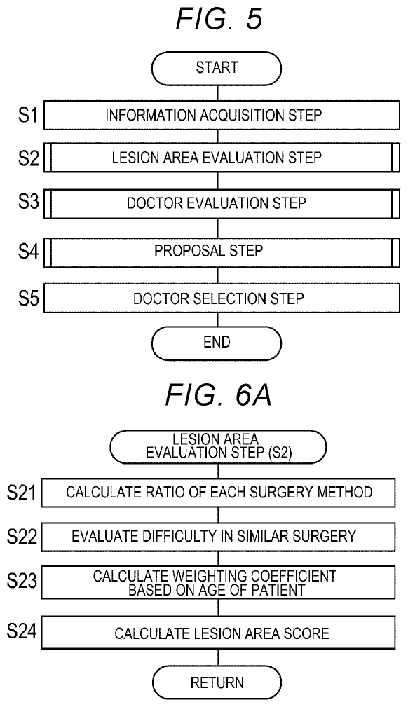

FIGS. 5, 6A to 6C, 7A, and 7B are views for describing an assistance method according to the present embodiment. Hereinafter, the assistance method according to the present embodiment will be described with reference to FIGS. 5, 6A to 6C, 7A, and 7B.

Referring to FIG. 5, the assistance method can include an information acquisition step S1, a lesion area evaluation step S2, a doctor evaluation step S3, a proposal step S4, and a doctor selection step S5. Hereinafter, each step will be described.

First, the information acquisition step S1 will be described.

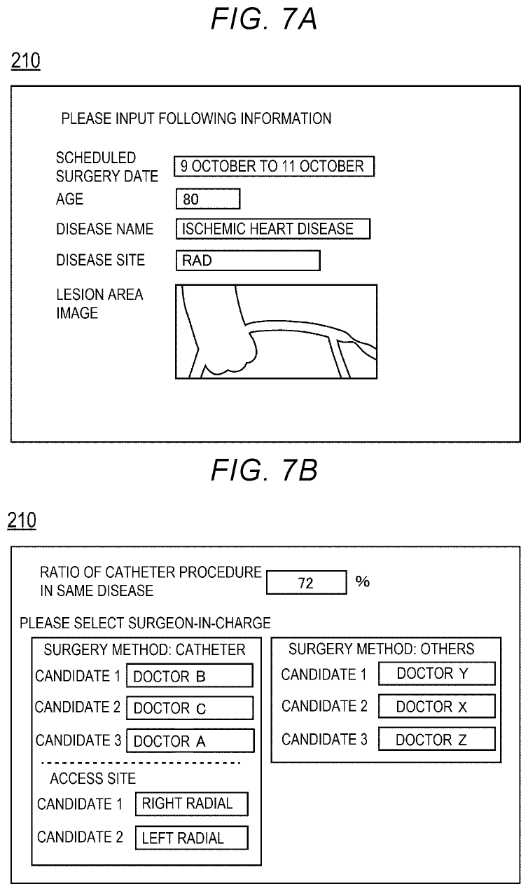

First, as illustrated in FIG. 1, the information acquisition unit 111 acquires surgery schedule information D3 relating to the scheduled surgery from the intra-hospital terminal 200 operated by the user of the assistance system 100. More specifically, as illustrated in FIG. 7A, the information acquisition unit 111 instructs the user to input the surgery schedule information via a display 210 of the intra-hospital terminal 200 operated by the user. The user of the assistance system 100 operates the intra-hospital terminal 200, and inputs information such as a scheduled surgery date, a patient name, a patient's age, a disease name, a diseases site, and lesion area information.

Next, as illustrated in FIG. 4A, the information acquisition unit 111 acquires the same disease surgery D10 from the past surgery information D1 stored in the storage unit 120.

Next, the lesion area evaluation step S2 will be described.

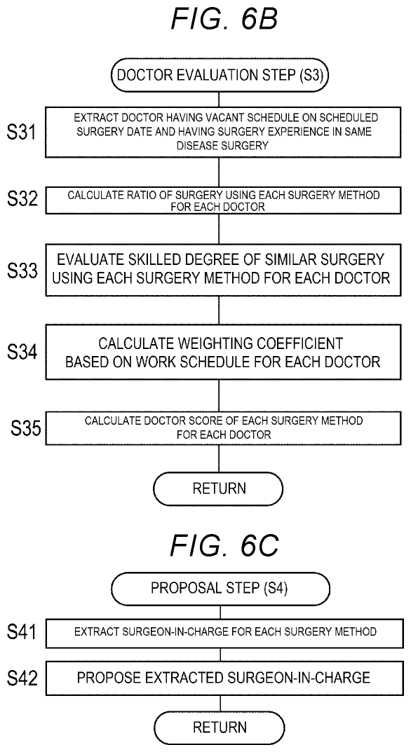

First, as illustrated in FIG. 6A, the surgery method analysis unit 112a classifies the same disease surgery D10 acquired by the information acquisition unit 111 for each surgery method, and calculates a ratio of the number of the surgeries using each surgery method to the number of the same disease surgeries D10 (Step S21). At this time, in a case of using the catheter surgery method, the surgery method analysis unit 112a may classify the same disease surgery D11 using the catheter for each access site, and may calculate the number of days required for patient discharge for each access site.

Next, the lesion area score calculation unit 112b evaluates the difficulty in the similar surgery for each surgery method (Step S22). Specifically, the lesion area score calculation unit 112b acquires the similar surgery from the same disease surgeries for each surgery method. For example, as expressed in Equation 1 above, for each surgery method, the lesion area score calculation unit 112b calculates a ratio of the average surgery time in the similar surgeries to the average surgery time in the same disease surgeries, as the difficulty in the similar surgery.

Next, the lesion area score calculation unit 112b calculates the weighting coefficient based on the age of the patient having the scheduled surgery (Step S23).

Next, the lesion area score calculation unit 112b calculates the difficulty in the scheduled surgery as the lesion area score, based on the difficulty in the similar surgeries and the age of the patient having the scheduled surgery for each surgery method (Step S24). Specifically, for example, as expressed in Equation 2 above, a value obtained by multiplying the difficulty in the similar surgery for each surgery method by the weighting coefficient based on the age of the patient having the scheduled surgery is calculated as the lesion area score which predicts the difficulty in the scheduled surgery (Step S24).

Next, the doctor evaluation step S3 will be described.

First, as illustrated in FIG. 6B, the doctor analysis unit 113a extracts the intra-hospital doctor having surgery experience in the same disease surgery D10 and having availability on the scheduled surgery date (Step S31).

Next, the doctor analysis unit 113a classifies the same disease surgery for each surgery method and for each doctor extracted in Step S31, and calculates a ratio of the number of the same disease surgeries using each surgery method to the number of the same disease surgeries (Step S32).

Next, the doctor score calculation unit 113b evaluates the skilled level of the similar surgeries using each surgery method for each doctor extracted in Step S31 (Step S33). Specifically, the doctor score calculation unit 113b acquires the similar surgery having the lesion area information similar to that of the scheduled surgery, from the same disease surgeries D10 acquired by the information acquisition unit 111. The doctor score calculation unit 113b classifies the acquired similar surgeries for each surgery method. For example, as expressed in Equation 3 above, the doctor score calculation unit 113b calculates a ratio of the average time in the similar surgeries of each doctor to the average surgery time in the similar surgeries for each surgery method, as the skill level of each doctor in the similar surgeries.

Next, the doctor score calculation unit 113b calculates the weighting coefficient based on the work schedule of each doctor (Step S34).

Next, the doctor score calculation unit 113b calculates the doctor score, based on the skilled level in the similar surgeries and the work schedule of each doctor for each surgery method (Step S35). Specifically, for example, as expressed in Equation 4 above, the doctor score calculation unit 113b calculates a value obtained by multiplying the skill level of each doctor in the similar surgeries by the weighting coefficient based on the work schedule, as the doctor score which predicts the capability of the doctor A on the scheduled surgery date.

Next, the proposal step S4 will be described.

First, as illustrated in FIG. 6C, for each surgery method, the proposal unit 114 extracts the doctor whose doctor score falls within a range of the upper limit value and the lower limit value based on the lesion area score, as the surgeon-in-charge (Step S41).

Next, the proposal unit 114 presents the ratio of each surgery method in the same disease surgeries D10 and the list of the doctors extracted as the surgeon-in-charge in each surgery method, to the user of the assistance system 100 (Step S42). At this time, in a case of using the catheter surgery method, the proposal unit 114 may present the access sites to the user, sequentially from the fewest (i.e., smallest) number of days required for patient discharge. Specifically, for example, as illustrated in FIG. 7B, the proposal unit 114 causes the display 210 of the intra-hospital terminal 200 operated by the user to display the ratio of the catheter surgery in the same disease surgeries and the list of the doctors extracted as the surgeon-in-charge. In a case of using the catheter surgery method, the access site is displayed, sequentially from the fewest number of days required for patient discharge.

Next, the doctor selection step S5 will be described.

First, the user of the assistance system 100 selects the surgeon-in-charge from the list of doctors presented in Step S42.

Next, the proposal unit 114 notifies the selected doctor, for example, by email that he or she is the surgeon-in-charge, and registers the scheduled surgery in the work schedule of the selected doctor.

After the surgery is actually performed, the storage unit 120 adds the surgery information on the performed surgery to the past surgery information D1.

As described above, the assistance system 100 according to the present embodiment supports the selection of the surgeon-in-charge. The assistance system 100 includes the information acquisition unit 111 that acquires the disease information, the lesion area information, and the scheduled surgery date of the scheduled surgery, the lesion area evaluation unit 112 that acquires the surgery information on the past similar surgery having the lesion area information similar to that of the scheduled surgery, that evaluates the difficulty in the similar surgery by using the surgery information on the past similar surgery, and that calculates the lesion area score which predicts the difficulty in the scheduled surgery, based on the evaluation result of the difficulty in the similar surgery, the doctor evaluation unit 113 that acquires the surgery information on the same past disease surgery previously performed by each of the plurality of doctors and having at least the disease information coinciding with that of the scheduled surgery, and each work schedule of the plurality of doctors, and that calculates the doctor score which predicts capability of each of the plurality of doctors at the scheduled surgery date by using the surgery information on the same past disease surgery and the work schedule, and the proposal unit 114 that proposes the doctor whose doctor score falls within the range of the upper limit value and the lower limit value based on the lesion area score, as the surgeon-in-charge.

According to the assistance system 100, the lesion area score which predicts the difficulty in the scheduled surgery can be calculated using surgery information on the past similar surgeries. The doctor score which predicts the capability of the doctor on the scheduled surgery date is calculated using the surgery information on the same past disease surgery and the work schedule. The doctor whose doctor score falls within the range of the upper limit value and the lower limit value based on the lesion area score is proposed as the surgeon-in-charge. That is, the doctor whose doctor score (capability on the scheduled surgery date) falls within a prescribed range with respect to the lesion area score (difficulty in the surgery) is proposed as the surgeon-in-charge. Therefore, according to the assistance system 100, the proper doctor can be proposed from the plurality of doctors in accordance with the physical condition of the doctor on the scheduled surgery date and the difficulty in the surgery. Therefore, the user can rather efficiently select the proper doctor.

The past surgery information includes the surgery time, and the lesion area evaluation unit 112 evaluates the difficulty in the similar surgery by comparing the average surgery time in the same past disease surgeries having the disease information coinciding with that of the scheduled surgery with the average surgery time in the past similar surgeries having the lesion area information similar to that of the scheduled surgery. Therefore, the lesion area evaluation unit 112 can evaluate how difficult it is to perform the similar surgery having the lesion area information similar to that of the scheduled surgery, even in the same disease surgery, and can calculate the lesion area score which predicts the difficulty in the scheduled surgery, based on the evaluation result.

The information acquisition unit 111 acquires the age of the patient having the scheduled surgery, and the lesion area evaluation unit 112 calculates the lesion area score, based on the difficulty in the similar surgery and the age of the patient having the scheduled surgery. Therefore, it is possible to predict the difficulty in the scheduled surgery in view of the age of the patient having the scheduled surgery.

The past surgery information includes the surgery time, and the doctor evaluation unit 113 calculates the doctor score of one doctor, based on the comparison result between the average surgery time in the same past disease surgeries having at least the disease information coinciding with that of the scheduled surgery, and the average surgery time in the surgeries performed by one doctor out of the plurality of doctors in the same past disease surgeries. Therefore, it is possible to evaluate the capability of each doctor in at least the same disease surgeries.

The assistance method according to the present embodiment assists the selection of the surgeon-in-charge. The assistance method includes the information acquisition step S1 of acquiring the disease information on the scheduled surgery, the lesion area information, and the scheduled surgery date, the lesion area evaluation step S2 of acquiring the surgery information on the past similar surgery having the lesion area information similar to that of the scheduled surgery, evaluating the difficulty in the similar surgery by using the surgery information on the past similar surgery, and calculating the lesion area score which predicts the difficulty in the scheduled surgery, based on the evaluation result of the difficulty in the similar surgery, the doctor evaluation step S3 of acquiring the surgery information on the same past disease surgery previously performed by each of the plurality of doctors and having at least the disease information coinciding with that of the scheduled surgery, and each work schedule of the plurality of doctors, and calculating the doctor score which predicts capability of each of the plurality of doctors at the scheduled surgery date by using the surgery information on the same past disease surgery and the work schedule, and the proposal step S4 of proposing the doctor whose doctor score falls within the range of the upper limit value and the lower limit value based on the lesion area score, as the surgeon-in-charge.

The assistance program according to the present embodiment assists the selection of the surgeon-in-charge. The assistance program can include the procedure of acquiring the disease information on the scheduled surgery, the lesion area information, and the scheduled surgery date, the lesion area evaluation procedure of acquiring the surgery information on the past similar surgery having the lesion area information similar to that of the scheduled surgery, evaluating the difficulty in the similar surgery by using the surgery information on the past similar surgery, and calculating the lesion area score which predicts the difficulty in the scheduled surgery, based on the evaluation result of the difficulty in the similar surgery, the procedure of acquiring the surgery information on the same past disease surgery previously performed by each of the plurality of doctors and having at least the disease information coinciding with that of the scheduled surgery, and each work schedule of the plurality of doctors, and calculating the doctor score which predicts capability of each of the plurality of doctors at the scheduled surgery date by using the surgery information on the same past disease surgery and the work schedule, and the procedure of proposing the doctor whose doctor score falls within the range of the upper limit value and the lower limit value based on the lesion area score, as the surgeon-in-charge.

The recording medium MD according to the present embodiment is a computer-readable recording medium having the assistance program recorded on the computer-readable recording medium MD.

According to the assistance method, the assistance program, and the recording medium MD having the assistance program recorded on the recording medium MD, the lesion area score which predicts the difficulty in the scheduled surgery is calculated using the surgery information on the past similar surgery. The doctor score which predicts the capability of the doctor on the scheduled surgery date is calculated using the surgery information on the same past disease surgery and the work schedule. The doctor whose doctor score falls within the range of the upper limit value and the lower limit value based on the lesion area score is proposed as the surgeon-in-charge. That is, the doctor whose doctor score (capability on the scheduled surgery date) falls within a prescribed range with respect to the lesion area score (difficulty in the surgery) is proposed as the surgeon-in-charge. Therefore, the assistance method, the assistance program, and the recording medium MD having the assistance program recorded on the recording medium MD can propose the proper doctor from the plurality of doctors in accordance with the physical condition of the doctor on the scheduled surgery date and the difficulty in the surgery. Therefore, the user can rather efficiently select the proper doctor.

Hitherto, the assistance system, the assistance method, the assistance program, and the recording medium having the assistance program recorded on the recording medium according to the present disclosure have been described with reference to the embodiment. However, the present disclosure is not limited to only the respectively described configurations, and can be appropriately modified based on the appended claims.

For example, means and methods for performing various processes in the assistance system may be realized by either a dedicated hardware circuit or a programmed computer. The assistance program may be provided online via a network such as the Internet.

The assistance system according to the present disclosure is not limited to a system which proposes the surgeon-in-charge for a cardiovascular disease, as in the assistance system according to the above-described embodiment. For example, the assistance system according to the present disclosure may propose the surgeon-in-charge for a lower limb artery disease or the surgeon-in-charge for a peripheral vascular disease.

The past surgery information may include information relating to a patient's condition such as a patient's blood pressure and an electrocardiogram during the surgery, and information relating to troubles occurring during the surgery and after the surgery. In this case, for example, the doctor score calculation unit may evaluate the skill level of the doctor in view of the information relating to the patient's condition and/or the information relating to the troubles.

The surgery schedule information may include a treatment history of the patient or a risk factor belonging to the patient. The risk factor can mean a factor that may cause another disease different from the surgery-scheduled disease (for example, hypertension or diabetes). For example, the information acquisition unit can acquire the treatment history and the risk factor of the patient from a patient's electronic medical record stored in the storage unit. The proposal unit may cause the display of the operation terminal operated by the user of the assistance system to display the risk factor together with the list of the doctors extracted as the surgeon-in-charge. In this manner, the user of the assistance system can select the more proper surgeon-in-charge in view of the risk factor.

The work schedule may be configured to have a schedule table including not only a daily work status (for example, outside duty, vacation, or on duty) of the doctors but also hourly work contents (for example, conference, office work, surgery, outpatient care, or night duty) of the doctors. In this case, for example, the doctor score calculation unit may set the weighting coefficient based on the work schedule to 1 or higher, before the scheduled surgery date, in a case where the doctor continuously performs the surgery for a predetermined number of days, or in a case where the doctor is on night duty on the previous day.

The method of calculating the lesion area score and the doctor score is not limited to the above-described method. For example, the lesion area score may be determined, based on a difference between the average surgery time in the same disease surgeries and the average surgery time in the similar surgeries. For example, the doctor score may be determined, based on a difference between the average surgery time in the same disease surgeries or the similar surgeries and the average surgery time of each doctor in the same disease surgeries or the similar surgeries. For example, the lesion area score may be determined to have any value from 0 to 100 points, based on a ratio or a difference between the average surgery time in the same disease surgeries and the average surgery time in the similar surgeries. For example, the doctor score may be determined to have any value from 0 to 100 points, based on a ratio or a difference between the average surgery time in the same disease surgeries or the similar surgeries and the average surgery time of each doctor in the same disease surgeries or the similar surgeries.

The assistance system may be connected to a terminal of another hospital or a server of a regional medical care center via an external network. In this case, the storage unit may store the surgery information on the past surgery performed by a doctor at another hospital, and the lesion area evaluation unit and the doctor evaluation unit may calculate the lesion area score and the doctor score by using the surgery information on the past surgery performed by a doctor at another hospital.

In the above-described embodiment, the proposal unit presents the access points, sequentially from the smallest number of days required for patient discharge. However, the proposal unit may present the access points, sequentially from the largest number of cases.

The detailed description above describes embodiments of an assistance system, an assistance method, an assistance program, and a recording medium having an assistance program recorded on the recording medium, which assist selection of a surgeon-in-charge for a scheduled surgery. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. An assistance system for assisting selection of a surgeon-in-charge from a plurality of doctors, the system comprising:
an information acquisition unit configured to acquire disease information, lesion area information, and a scheduled surgery date of a scheduled surgery;
a lesion area evaluation unit configured to acquire surgery information on a past similar surgery having the lesion area information similar to that of the scheduled surgery, configured to evaluate a difficulty in the past similar surgery by using the surgery information on the past similar surgery, and configured to calculate a lesion area score which predicts a difficulty in the scheduled surgery, based on an evaluation result of the difficulty in the past similar surgery;
a doctor evaluation unit configured to acquire surgery information on a same past disease surgery previously performed by each of the plurality of doctors and having at least disease information coinciding with that of the scheduled surgery, and each work schedule of the plurality of doctors, and configured to calculate a doctor score which predicts capability of each of the plurality of doctors at the scheduled surgery date by using the surgery information on the same past disease surgery and the work schedule; and
a proposal unit configured to propose the doctor whose doctor score falls within a range of an upper limit value and a lower limit value based on the lesion area score, as the surgeon-in-charge for the scheduled surgery.

2. The assistance system according to claim 1,
wherein the past surgery information includes a surgery time; and
wherein the lesion area evaluation unit is configured to evaluate the difficulty in the past similar surgery by comparing an average surgery time of the same past disease surgery having the disease information coinciding with that of the scheduled surgery, with an average surgery time of the past similar surgery having the lesion area information similar to that of the scheduled surgery.

3. The assistance system according to claim 2,
wherein the information acquisition unit is configured to acquire an age of a patient of the scheduled surgery; and
wherein the lesion area evaluation unit is configured to calculate the lesion area score based on the difficulty in the past similar surgery and the age of the patient of the scheduled surgery.

4. The assistance system according to claim 1,
wherein the past surgery information includes a surgery time; and
wherein the doctor evaluation unit is configured to calculate the doctor score of one doctor, based on a comparison result between an average surgery time of the same past disease surgery having at least the disease information coinciding with the scheduled surgery, and an average surgery time of a surgery performed by the one doctor out of the plurality of doctors in the same past disease surgery.

5. The assistance system according to claim 1, wherein the doctor evaluation unit is configured to proposing the surgeon-in-charge to be the doctor having availability on the scheduled surgery date with a shortest surgery time for the same past disease surgery.

6. The assistance system according to claim 1, further comprising:
a terminal configured to display the proposed doctor as the surgeon-in-charge.

7. An assistance method for assisting selection of a surgeon-in-charge from a plurality of doctors, the method comprising:
acquiring disease information on a scheduled surgery, lesion area information, and a scheduled surgery date;
acquiring surgery information on a past similar surgery having the lesion area information similar to that of the scheduled surgery, evaluating a difficulty in the past similar surgery by using the surgery information on the past similar surgery, and calculating a lesion area score which predicts a difficulty in the scheduled surgery, based on an evaluation result of the difficulty in the past similar surgery;
acquiring surgery information on the same past disease surgery previously performed by each of the plurality of doctors and having at least disease information coinciding with that of the scheduled surgery, and each work schedule of the plurality of doctors, and calculating a doctor score which predicts capability of each of the plurality of doctors at the scheduled surgery date by using the surgery information on the same past disease surgery and the work schedule; and
proposing the doctor whose doctor score falls within a range of an upper limit value and a lower limit value based on the lesion area score, as the surgeon-in-charge for the scheduled surgery.

8. The assistance method according to claim 7, wherein the past surgery information includes a surgery time, the method comprising:
evaluating the difficulty in the past similar surgery by comparing an average surgery time of the same past disease surgery having the disease information coinciding with that of the scheduled surgery, with an average surgery time of the past similar surgery having the lesion area information similar to that of the scheduled surgery.

9. The assistance method according to claim 8, comprising:
acquiring an age of a patient of the scheduled surgery; and
calculating the lesion area score based on the difficulty in the past similar surgery and the age of the patient of the scheduled surgery.

10. The assistance method according to claim 7, wherein the past surgery information includes a surgery time, the method comprising:
calculating the doctor score of one doctor, based on a comparison result between an average surgery time of the same past disease surgery having at least the disease information coinciding with the scheduled surgery, and an average surgery time of a surgery performed by the one doctor out of the plurality of doctors in the same past disease surgery.

11. The assistance method according to claim 7, further comprising:
displaying the proposed doctor as the surgeon-in-charge on a terminal.

12. The assistance method according to claim 7, wherein the past surgery information includes a patient age, disease information, the lesion area information, a surgery method, an access site, a surgery time, and a number of days required for patient discharge, and a surgeon-in-charge, the method comprising:
classifying the surgery method as a surgery using a catheter and a surgery performed using a method other than the catheter; and
further classifying the same disease surgery based on the surgery method.

13. The assistance method according to claim 7, comprising:
proposing the surgeon-in-charge to be the doctor of the plurality of doctors having a shortest surgery time for the same past disease surgery.

14. A non-transitory computer readable medium (CRM) storing computer program code executed by a computer processor that executes a process for assisting selection of a surgeon-in-charge from a plurality of doctors, the process comprising:
acquiring disease information on a scheduled surgery, lesion area information, and a scheduled surgery date;
acquiring surgery information on a past similar surgery having the lesion area information similar to that of the scheduled surgery, evaluating a difficulty in the similar surgery by using the surgery information on the past similar surgery, and calculating a lesion area score which predicts a difficulty in the scheduled surgery, based on an evaluation result of the difficulty in the similar surgery;
acquiring surgery information on the same past disease surgery previously performed by each of the plurality of doctors and having at least disease information coinciding with that of the scheduled surgery, and each work schedule of the plurality of doctors, and calculating a doctor score which predicts capability of each of the plurality of doctors at the scheduled surgery date by using the surgery information on the same past disease surgery and the work schedule; and
proposing the doctor whose doctor score falls within a range of an upper limit value and a lower limit value based on the lesion area score, as the surgeon-in-charge for the scheduled surgery.

15. The computer readable medium according to claim 14, wherein the past surgery information includes a surgery time, the process comprising:
evaluating the difficulty in the past similar surgery by comparing an average surgery time of the same past disease surgery having the disease information coinciding with that of the scheduled surgery, with an average surgery time of the past similar surgery having the lesion area information similar to that of the scheduled surgery.

16. The computer readable medium according to claim 15, comprising:
acquiring an age of a patient of the scheduled surgery; and
calculating the lesion area score based on the difficulty in the past similar surgery and the age of the patient of the scheduled surgery.

17. The computer readable medium according to claim 14, wherein the past surgery information includes a surgery time, the process comprising:
calculating the doctor score of one doctor, based on a comparison result between an average surgery time of the same past disease surgery having at least the disease information coinciding with the scheduled surgery, and an average surgery time of a surgery performed by the one doctor out of the plurality of doctors in the same past disease surgery.

18. The computer readable medium according to claim 14, further comprising:
displaying the proposed doctor as the surgeon-in-charge on a terminal.

19. The computer readable medium according to claim 14, wherein the past surgery information includes a patient age, disease information, the lesion area information, a surgery method, an access site, a surgery time, and a number of days required for patient discharge, and a surgeon-in-charge, the process comprising:
classifying the surgery method as a surgery using a catheter and a surgery performed using a method other than the catheter; and
further classifying the same disease surgery based on the surgery method.

20. The computer readable medium according to claim 14, comprising:
proposing the surgeon-in-charge to be the doctor of the plurality of doctors having a shortest surgery time for the same past disease surgery.
